(19) United States
(12) Patent Application Publication
(10) Pub. No.: US 2008/0319278 A1
(43) Pub. Date: Dec. 25, 2008

(54) Sensor

(75) Inventors: Tore Omtveit, Eiksmarka (NO); Ann Kjersti Fahlvik, Oslo (NO); Peyman Mirtaheri, Oslo (NO); Tor Inge Tonnessen, Oslo (NO)

Correspondence Address: CANTOR COLBURN, LLP, 20 Church Street, 22nd Floor, Hartford, CT, 06103, US

(73) Assignee: ALERTIS MEDICAL AS, Oslo (NO)

(21) Appl. No.: 11/574,852

(22) PCT Filed: Sep. 8, 2005
(86) PCT No.: PCT/GB05/03461

§ 371 Date: Mar. 11, 2008

(30) Foreign Application Priority Data
Sep. 8, 2004 (GB) 0419958.4

(51) Int. Cl. A61B 5/02

(52) U.S. Cl. 600/301

(57) ABSTRACT

A physiological sensing device comprises, in combination a sensor (4) for the measurement of the partial pressure of carbon dioxide (pCO₂), a body temperature sensor (5) and a heart rate and oxygen saturation sensor (54). The sensor device can be used to continuously monitor the vital signs of a patient.

The invention relates to a physiological sensor.

A simple sensor particularly suitable for partial pressure of carbon dioxide (pCO₂) measurement, especially as part of a technique for monitoring for ischemias, is described in WO 00/04386.

In addition to the detection of ischemia, it has now been realised that the measurement of pCO₂ may be useful in the diagnosis of severe and potentially life threatening conditions leading to changes in e.g. blood perfusion of tissues, respiration and/or the metabolism, such as shock and sepsis. Thus, it would be advantageous to provide a sensing device which is particularly suited to the monitoring of the hospitalised patient, also outside intensive care units, to detect the onset of sepsis.

Viewed from a first aspect, the present invention provides a physiological sensing device comprising in combination:

a sensor for the measurement of the partial pressure of carbon dioxide (pCO₂);

a body temperature sensor;

a heart rate sensor; and

an oxygen saturation sensor.

Thus, according to the invention a single device can be provided which measures key vital signs such as pCO₂, body temperature, pulse and blood oxygenation. It is believed that the measurement and monitoring of just these four parameters allows a physician to identify the onset of critical and treatment-requiring conditions in a patient such as, for example, sepsis. Consequently, the device according to the invention allows a physician to conveniently and accurately monitor a patient for the onset of sepsis.

In general, the pCO₂ sensor is configured for insertion through a patient's skin. In this way, the sensor may be inserted into the tissue, for example a muscle, of the patient. Thus, the sensor may be dimensioned for insertion into the tissue of a patient with minimal disruption to the tissue. The pCO₂ sensor may be configured to penetrate the patient's skin (and tissue). Consequently, the pCO₂ sensor or the device in general, may be provided with a sharp, for example pointed, tip. Alternatively, the pCO₂ sensor may be configured for insertion into an incision in the patient's tissue.

Viewed from a further aspect, therefore, the invention also provides a physiological sensing device comprising a pCO₂ sensor configured for insertion through a patient's skin and a sharp tip for puncturing a patient's skin on insertion of the pCO₂ sensor.

The sensor device may be provided with an insertion device for inserting the pCO₂ sensor through the patient's skin. In one embodiment, the insertion device is a removable mandrel which is received in a sheath connected to the pCO₂ sensor and engages the pCO₂ sensor to force it through the patient's skin. The mandrel may be removed once the pCO₂ sensor has been inserted in the patient's tissue.

Alternatively, the sensor device may comprise a hollow needle in which the pCO₂ sensor is received for insertion through a patient's skin. The hollow needle may be removable from the sensor device after insertion of the pCO₂ sensor. Advantageously, the cross-section of the needle may be an open curve. This has the advantage that the electrical connections to the pCO₂ sensor can pass through the needle and can be separated from the needle when the needle is removed from the patient. For example, the needle may has a cross-section that is U-shaped, V-shaped or C-shaped.

Advantageously, the device is provided with a self-sealing membrane to close the hole for the needle (or other insertion device) when the needle is removed.

Advantageously, the sensor device and/or the insertion device may be provided with disinfectant, particularly on the pCO₂ sensor, temperature sensor or sharp tip, in order that the sensor device can be applied quickly to a patient, for example in an emergency. Thus, the sensor device may be packaged with disinfectant on those surfaces that will contact the patient.

The pCO₂ sensor may be connected to an electrical cable for communicating signals from the sensor and connected electrically at its distal end to the sensor. The device may comprise a sheath mechanically connected to the pCO₂ sensor and extending with and surrounding at least a portion of the length of the cable. In one arrangement, the sheath comprises a plurality of substantially longitudinally extending flexible portions separated by a plurality of longitudinal slits, such that movement of the proximal end of the sheath towards the distal end of the sheath shortens the distance between the ends of the flexible portions and causes the flexible portions to project outwardly and thereby increase the effective diameter of the sheath in the region of the flexible portions, such that the pCO₂ sensor can be retained in tissue by the projecting flexible portions.

Thus, according to this arrangement, the sensor can be inserted into the patient's tissue and the cable can be pulled to draw the ends of the flexible portions together and cause them to project outwardly. The projecting flexible portions engage with the patient's tissue and retain the pCO₂ sensor in position while the sensor monitors the physiology of the organ. When monitoring is complete, the proximal end of the sheath can be released so that the flexible portions return to their original position flush with the sheath and disengage the tissue. The sensor can then be removed easily from the patient.

The flexible portions may be resilient, for example composed of a resilient material. The flexible portions may be biased into the flush position, for example by their own resilience or by a separate resilient component.

A locking mechanism may be provided, for example at the proximal end of the sheath, to maintain the ends of the sheath in the position in which the flexible members project outwardly.

The device may further comprise a line, for example a Kevlar line, which is mechanically connected to the distal end of the sheath. The line may extend longitudinally with the cable to assist in pulling the distal end of the sheath towards the proximal end of the sheath. Such a line has the advantage that it is not necessary for the cable and/or the electrical connections to the sensor to be strong enough to withstand the forces necessary to bow the flexible members.

It is possible that the cable may be surrounded by a further conduit in addition to the sheath, but this is not preferred. In a simple embodiment, the cable is surrounded only by the sheath.

Advantageously, the sheath may form a carbon dioxide permeable membrane of the pCO₂ sensor. This provides a particularly simple construction. Suitable materials for the sheath in this case are PTFE, silicone rubbers and polyolefins.

The sensor device may be provided with an attachment portion for attaching the device to the surface of the patient's skin. In one convenient embodiment, the attachment portion is an adhesive patch, such as a plaster. In the context of a pCO₂ sensor, this is believed to be a novel aspect of the invention. Thus, viewed from a further aspect, the invention provides a physiological sensing device comprising a pCO₂ sensor configured for insertion through a patient's skin and an adhesive patch for adhering the device to a patient's skin to retain the inserted pCO₂ sensor in position.

The provision of a plaster, as well as retaining the sensor device in position, has several other advantages. In particular, the plaster seals the point at which the pCO₂ sensor is inserted through the patient's skin, thereby reducing the risk of infection. In this regard, the patient-facing side of the plaster may be provided with disinfectant or antibiotics. Furthermore, the plaster may conveniently carry wires, other sensors or a wireless communication device.

Such a device is conveniently applied to the patient and retained in position while the patient is monitored. Desirably, the electrical and mechanical connections to the pCO₂ sensor, such as electrical cables and sheaths are flexible. In this way, the discomfort to the patient when the pCO₂ sensor has been inserted is minimised.

The sensor may comprise a closed chamber bounded, at least partially, by a carbon dioxide permeable membrane; and at least two electrodes within the chamber, with the chamber containing substantially electrolyte-free liquid in contact with the electrodes and the membrane.

By substantially electrolyte-free, it is meant that the liquid has an ionic osmolality no greater than that at 37° C. of an aqueous 5 mM sodium chloride solution, preferably no more than that of a 500 μM sodium chloride solution, more especially no more than that of a 10⁻⁵ to 10⁻⁶ M HCl solution.

Preferably, the liquid in contact with the electrodes is aqueous and especially preferably it is water, substantially electrolyte-free as defined above. Other solvents that react with CO₂ to increase or decrease their conductance, e.g. by the production or neutralization of ions, may likewise be used. In practice, however, deionized or distilled water with or without the addition of a strong acid (e.g. HCl) to a concentration of 0.1 to 100 μM, preferably 0.5 to 50 μM, more especially about 1 μM, has been found to function particularly well. The function of this small addition of acid is generally to maintain the pH of the liquid at 6 or below to avoid significant contributions to conductance by hydroxyl ions and to maintain the linearity of the measurements of pCO₂.

The liquid may contain a non-ionic excipient. In this way, the osmolarity of the liquid in the chamber can be increased to prevent egress of the liquid across the membrane, without affecting the electrical characteristics of the liquid.

The excipient should have at least isotonic concentration, i.e. should be isosmotic with an aqueous solution of 0.9% w/v NaCl. Preferably, the concentration of the excipient is hypertonic, i.e. is hyperosmotic with 0.9% w/v aqueous NaCl. Thus, the osmolality of the excipient in the chamber may be greater than that of 0.9% w/v aqueous NaCl, preferably greater than that of 1.8% w/v aqueous NaCl (twice isotonic concentration). Osmolalities greater than that of 4.5% w/v aqueous NaCl (five times isotonic concentration), or even greater than that of 9% w/v aqueous NaCl (ten times isotonic concentration) may be used.

Any suitable excipient may be used that is inert to the bicarbonate reaction in the chamber. The excipient should also be soluble in the liquid, for example water. The excipient is also desirably an accepted pharmaceutical excipient for intravenous use and with low viscosity for simple filling of the chamber. The excipient should preferably be sterilizable and storage stable. Desirably, the excipient should inhibit microbiological growth.

A suitable excipient is polyethylene glycol (PEG) and the presently preferred excipient is propylene glycol.

The primary components of the pCO₂ sensor are an electrode chamber, a CO₂-permeable membrane forming at least part of the wall of the electrode chamber, first and second electrodes having surfaces within said chamber (or providing internal surfaces to said chamber), and a liquid (generally substantially electrolyte-free water) in the electrode chamber in contact with the membrane and the first and second electrodes. The sensor includes or is connectable to an AC power supply, a conductance (or resistance) determining device, a signal generator (which may be part of the determining means) and optionally a signal transmitter.

The mechanism by which pCO₂ is determined using the sensor device of the invention is straightforward. In a pure protic solvent, e.g. water, the electrical resistance is high because of the paucity of ionic species. Addition of CO₂ results in formation (with water) of H⁺ and HCO⁻₃ ions and thus a reduction in the electrical resistance. Since the only factor responsible for reduction in resistance in the sensor is CO₂ passing through the membrane, the change in resistance enables pCO₂ to be measured.

From the equilibrium constant for the H₂O+CO₂ to H⁺+HCO⁻₃ equilibrium, CO₂ concentration is equal to αpCO₂ (where α at 25° C. is 0.310). The electrical conductivity for protons is G_{H+}=349.8 S.cm²/mol, that for hydroxyls is G_{OH-}=198.3 S.cm²/mol and that for bicarbonate is G_{HCO3-}=44.5 S.cm²/mol. The concentrations of H⁺ and OH⁻ vary inversely, and the concentrations of H⁺ and HCO₃⁻ are directly proportional to pCO₂. The total conductance of the solution is thus effectively proportional to pCO₂ since the contribution of OH⁻ is minimal. The conductivity of the solution Gₛₒₗᵤₜᵢₒₙ is thus given by

Gₛₒₗᵤₜᵢₒₙ=θ_{H+}[H⁺]G_{H+}+θ_{OH-}[OH⁻]G_{OH-}+θ_{HCO-3}[HCO₃⁻]G_{HCO3-}

where θ_{H-}, θ_{OH-} and θ_{HCO3-} are the activity coefficients for the three ionic species.

Table 1 below shows, by way of example, measured pCO₂ and pH values and corresponding calculated values for H⁺, OH⁻ and HCO₃⁻ concentrations showing the increase of H⁺ and HCO₃⁻ with increasing pCO₂.

| | | | [H⁺] | [OH] | [HCO₃] |
|---|---|---|---|---|---|
| Sample number | pCO₂ (kPa) | pH | (mmol/l) | (mmol/l) | (mmol/l) |
| 1 | 6.38 | 5.141 | 7.23E−06 | 1.38E−09 | 7.23E−06 |
| 2 | 9.64 | 5.060 | 8.71E−06 | 1.15E−09 | 8.71E−06 |
| 3 | 15.37 | 4.891 | 1.29E−05 | 7.78E−10 | 1.29E−05 |
| 4 | 25.88 | 4.760 | 1.74E−05 | 5.75E−10 | 1.74E−05 |
| 5 | 31.48 | 4.664 | 2.17E−05 | 4.61E−10 | 2.17E−05 |

(pCO₂ and pH measured with a standard blood gas analyser, ABL® System 625 at 37° C.)

The electrical conductivity is measured in the solvent film in the pCO₂ sensor of the invention. This can be done by applying a constant voltage (or current) to the electrodes and measuring the current (or voltage) changes which correspond to changes in conductivity as CO₂ enters the solvent through the membrane. Preferably however an alternating sine wave function voltage with a constant peak value is applied and the voltage drop across the electrodes is measured. The solution conductivity is then equal to the current passed through the electrode divided by the voltage drop across the electrodes.

The pCO₂ sensor may function by applying an alternating electrical potential to the electrodes whereby to cause an alternating current in the liquid. The liquid should be reactive with carbon dioxide to alter its conductance. The electrical potential may have a frequency of 20 to 10,000 Hz, preferably 100 to 4,000 Hz.

The pCO₂ sensors of the invention are provided with or are connectable to an electrical power source arranged to apply an alternating electrical potential across the electrodes with a frequency of 100 to 10,000 Hz. The frequency is preferably greater than 1 kHz. The frequency is preferably less than 5 kHz, more preferably less than 2 kHz. At frequencies below 100 Hz, the sensitivity of pCO₂ determination is lower due to electropolarization and moreover the instrument response time becomes overly slow, while at frequencies above 10 kHz sensitivity is again less due to the low impedance of the capacitances in the sensor.

The power source may be an AC power source or alternatively a DC source in conjunction with an oscillator, i.e. a combination which together constitutes an AC power source.

The power supply is preferably such that the maximum current density through the liquid at the electrodes is no more than 50 A/m², preferably no more than 30 A/m², more preferably no more than 20 A/m², in particular no more than 10 A/m², and most preferably about 1 A/m² or below. Higher current density values of 20 A/m² or greater should only be used at the higher frequencies, e.g. 1-10 kHz. The smallest maximum current density is determined by detection limits, but values down to 10⁻⁸ A/m² are usable. The smallest maximum current density however will generally be at least 0.1 μA/m².

By operating at such current densities and voltage frequencies, and by appropriate construction, the sensor can determine the conductance/resistance of the liquid into which the CO₂ migrates without any significant loss of accuracy arising as a result of the electropolarization of the electrodes.

For particularly high accuracy, the potential or current across the electrodes (and hence the resistance or conductance of the liquid between the electrodes) is determined using a lock-in amplifier set to the same frequency as that of the voltage generator or electrical power source.

Furthermore it is preferred to incorporate in the detection a high pass filter to screen out current with a frequency less than 100 Hz, preferably less than 150 Hz. The filter is preferably a passive filter, for example a capacitor and a resistor.

The power source and the detector circuitry may, if desired, be included in the sensor of the invention. In this case, if it is desired that the sensor be wireless, it will preferably also be provided with means enabling the signal to be detected remotely, e.g. a transmitter, for example a RF transmitter.

A further electrode may be provided that is electrically connected to the patient, for example to the patient's skin. The signal from this further electrode may be processed with the signal from the sensor in order to compensate for electromagnetic noise from the patient.

Electropolarization effects are considerably reduced by increasing the surface area of the electrodes in contact with the liquid, e.g. by siting the electrodes in wells disposed away from the plane of the membrane or by using non-planar electrode surfaces, e.g. rough or textured surfaces. In general therefore it is desirable to have as large a ratio of surface area of electrode to liquid contact as possible, and as shallow as possible a liquid depth over as much as possible of its area of contact with the membrane. In this way the response time is reduced, electropolarization is reduced, lower frequencies may be used and stray capacitance effects are considerably reduced.

Increased electrical resistance relative to the resistance at the electrodes may be achieved by restricting the cross sectional area of the electrical path through the liquid between the electrodes at a zone in which the liquid is in contact with the membrane, e.g. by decreasing the depth of the liquid for a part of the path between the electrodes, and/or by ensuring a relatively large area of contact between each electrode and the liquid.

The resistance of the liquid at the membrane and between the electrodes may be increased by the use of structural elements to define liquid channels across the membrane between the electrodes, e.g. by disposing the membrane across or adjacent an insulating chamber wall portion in which such channels are formed, for example by etching. Likewise a porous spacer may be disposed between the membrane and the chamber wall to define the depth of the liquid.

Indeed, such spacers are important to use where, under the pressure conditions experienced in use, the membrane is sufficiently flexible and the liquid depth behind the membrane sufficiently small, for the measured conductance to vary with pressure.

In a preferred arrangement, the pCO₂ sensor comprises:

a sensor body having a longitudinal axis;

at least two electrodes spaced in a direction transverse to the longitudinal axis of the sensor body;

a plurality of support members extending outwardly from the axis of the sensor body and defining between adjacent support members at least one liquid channel that provides a fluid pathway between the electrodes; and

a gas-permeable membrane supported by the support members and providing an outer wall of the liquid channel(s).

This arrangement provides a compact configuration of the sensor with a longitudinal geometry that is suited to insertion in the tissue of a patient. Furthermore, the support members are able to provide physical support to the membrane, as well as defining liquid channels of small cross-sectional area that allow accurate measurement.

In order to reduce the electropolarisation effect mentioned above, the electrodes may be located in a recess in the sensor body that has a greater cross-sectional area than the liquid channels. In this way, the current density around the electrodes is reduced by the greater volume for liquid.

The electrodes of the pCO₂ sensor may extend longitudinally, for example parallel to the longitudinal axis of the sensor body.

Similarly, the liquid channel(s) may be transverse, for example perpendicular, to the longitudinal axis of the sensor body. In a preferred arrangement, the pCO₂ sensor comprises a plurality of liquid channels. For example, the sensor may comprise at least three liquid channels.

The support members may be transverse to the longitudinal axis of the sensor body. For example, the support members may be perpendicular to the longitudinal axis of the sensor body in the circumferential direction. In a preferred arrangement, the support members are in the form of rings formed about the longitudinal axis of the sensor body. The cross-section of the support members may be any suitable shape. It has been found in particular that support members with a substantially triangular, in particular sawtooth, cross-section are particularly easily formed by injection moulding. Alternatively, a substantially rectangular cross-section may be used. The support members may be formed integrally with the sensor body, for example by injection moulding. The sensor preferably comprises at least four support members.

The sensor body and/or the pCO₂ sensor may be generally cylindrical. The membrane may be arranged to surround the sensor body.

The described geometry may be applied to any suitable sensor. In the preferred arrangement, the sensor is a pCO₂ sensor.

Where the pCO₂ sensor is constructed with the liquid film in place, the electrodes are preferably of, or plated with, an inert material such that the resistivity of the liquid will not change significantly with storage. Suitable materials include platinum (especially black platinum), gold, silver, aluminium and carbon. Gold is particularly preferred. In general inert electrodes which do not generate solvated ions are preferred.

The membrane may be any material which is permeable to CO₂, and substantially impermeable to the solvent of the liquid, any electrolyte and water. Polytetrafluoroethylene, e.g. Teflon®, silicone rubber, polysiloxane, polyolefins or other insulating polymer films may be used, e.g. at thicknesses of 0.5 to 250 μm. The thicker the membrane, in general the slower the response time of the pCO₂ sensor will be. However the thinner the membrane the greater the risk of non-uniformities or of perforation or other damage. Conveniently, however, the thickness of the membrane will be 1 to 100 μm, preferably 50 to 100 μm.

The walls of the chamber of the pCO₂ sensor of the invention may be of any suitable material, e.g. plastics. Preferably the material should be capable of withstanding conditions normally used in sterilisation, e.g. radiation sterilization (for example using gamma radiation) or thermal sterilization (for example using temperatures of about 121° C. as used in autoclave sterilisation). In the case of thermal sterilization, the liquid will generally be sterile filled into the sensor after sterilization. The walls of the chamber and the membrane may be of the same material, e.g. Teflon®, machined to have self-supporting walls and a thinner gas-permeable membrane.

The pCO₂ sensor of the invention is generally relatively inexpensive and so, unlike prior art sensors, may be single-use devices. Moreover the electrode chamber can be made extremely small without difficulty (unlike the prior art glass electrode containing sensors for which miniaturization poses insuperable impedance problems).

The above arrangement provides a pCO₂ sensor, which can be inserted easily into the tissue of an animal, including a human, which can be retained in the tissue during monitoring and which can be removed easily when monitoring is complete.

The pCO₂ sensor is sufficiently small that it will not cause undue disturbance to the tissue to be monitored. Consequently, the sensor may have a maximum diameter of 2 mm, preferably 1 mm.

The temperature sensor may be applied to the patient's skin, in use of the sensor device. However, in one embodiment of the invention, the temperature sensor is configured for insertion through the patient's skin. In particular, the temperature sensor and the pCO₂ sensor may be incorporated into a single sensor unit. In other words, the pCO₂ sensor may include the temperature sensor.

Blood oxygen saturation levels may be measured by pulse oxymetry. Thus, the device may comprise a pulse oxymetry sensor. In pulse oxymetry, the saturation of oxyhaemoglobin in a patient's blood is determined by measuring the absorption of light by the haemoglobin. The degree of absorption differs depending on whether the haemoglobin is saturated or desaturated with oxygen. The blood oxygenation sensor according to the present invention may, in particular, be a reflectance pulse oxymetry sensor. In other words, the sensor may be configured to illuminate the patient's skin with light of a specified wavelength or wavelengths and measure the reflectance of these wavelengths in order to determine the degree of oxygen saturation of the patient's blood. Conveniently, therefore, the blood oxygenation sensor may be configured to be retained against the patient's skin by the adhesive patch.

The sensor device may comprise a dedicated heart rate sensor. Conveniently, however, the oxygen saturation sensor and heart rate sensor are provided by a pulse oxymetry sensor.

The sensor device may comprise a plurality of sensors for respective physiological parameters. For example, the device may comprise an array of sensors. Such sensors may measure one or more of the partial pressure of carbon dioxide, the partial pressure of oxygen, temperature, pH or glucose concentration, for example. The sensors may be provided, for example, on the plaster or adhesive patch. In the presently preferred embodiment, the device comprises a temperature sensor, a pCO₂ sensor, a heart rate sensor and a blood oxygenation sensor.

The pCO₂, oxygenation and temperature determined by the sensor device may be a quantified value or may simply be an indication that the values are above or below one or more threshold values indicative of sepsis, values which may be varied according to the location of the measurement site.

The sensor device may be used for a single measurement or, more preferably, may be used for continuous or repeated monitoring, e.g. in emergency and intensive care settings or in the ward or nursing homes of any risk patient for fast detection and immediate treatment of changes in vital signs.

Although the sensor has been described in relation to the detection of sepsis, it may be used to detect any condition that will cause either hypocarbia or hypercarbia in the tissue, i.e. any condition that will either change the respiratory pattern of the patient, or conditions that will increase the production of or reduce the elimination of CO₂. Conditions where hypocarbia is likely to be found include sepsis, fever of origin other than sepsis per se, moderate cardiac failure, pulmonary oedema, acute respiratory distress syndrome (ARDS) and hyperventilation of any cause. Conditions where hypercarbia is likely to be found include ischemia at the place where the sensor is located, circulatory shock of haemorrhagic, cardiac or septic origin and respiratory insufficiency, acute or chronic, such as ARDS or chronic obstructive lung disease (COLD).

An embodiment of the invention will now be described, by way of example only, with reference to the accompanying drawings, in which:

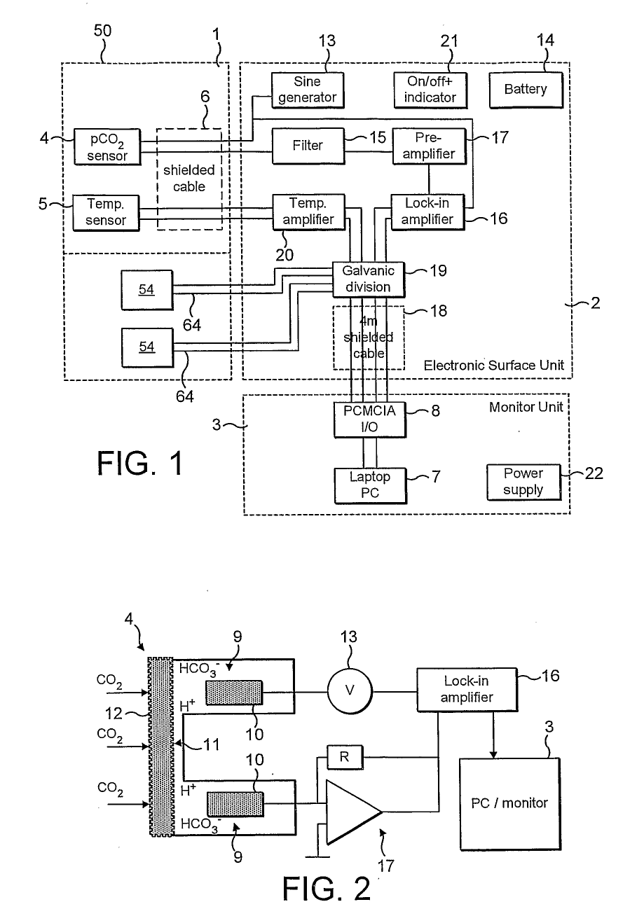

FIG. 1 is a schematic diagram of a complete sensing system incorporating the sensor device of the invention;

FIG. 2 is a schematic diagram illustrating the measurement principle for the pCO₂ sensor in the system of FIG. 1;

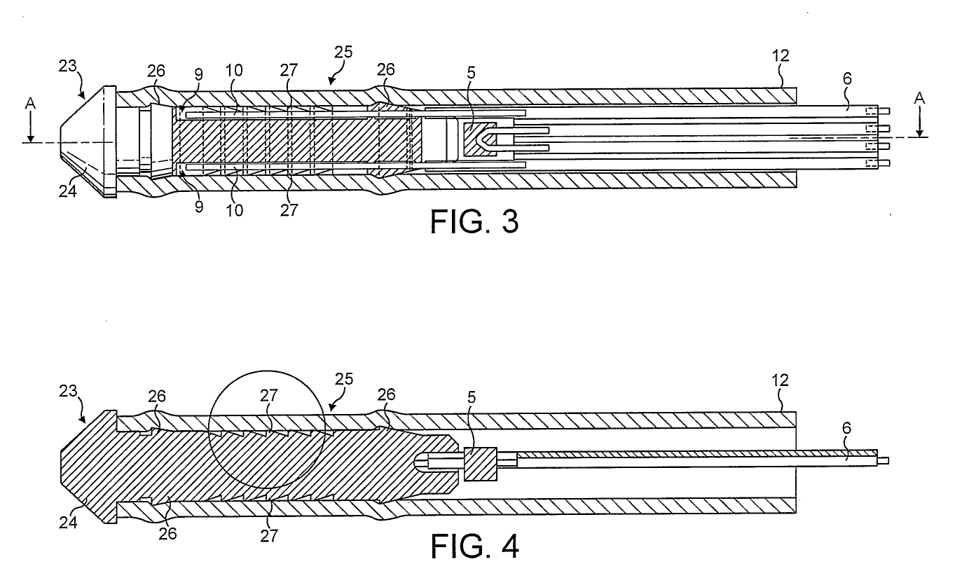

FIG. 3 is a partially cutaway view of a pCO₂ sensor according to the invention;

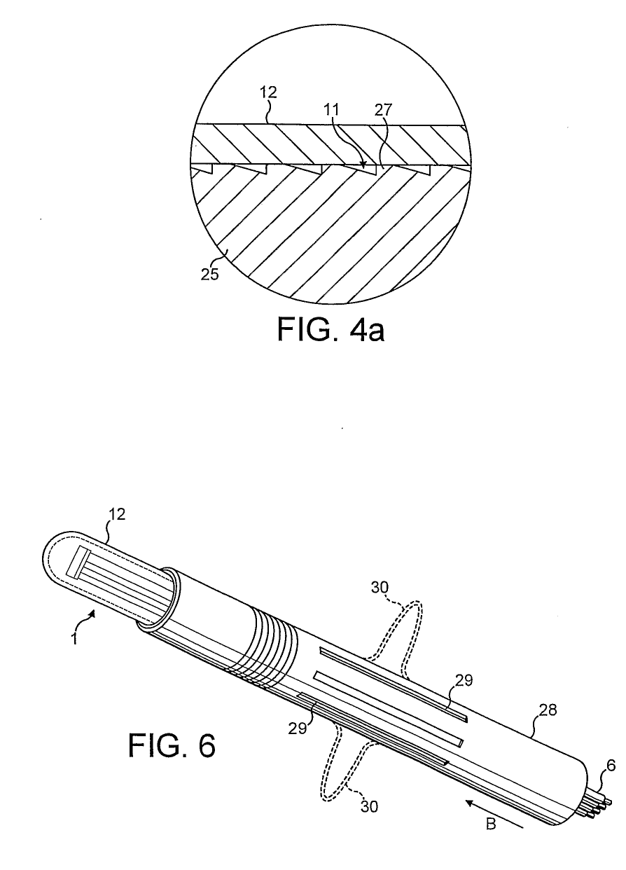

FIG. 4 is a cross-sectional view along line A-A of FIG. 3;

FIG. 4a is a magnified view of the detail indicated by the circle in FIG. 4;

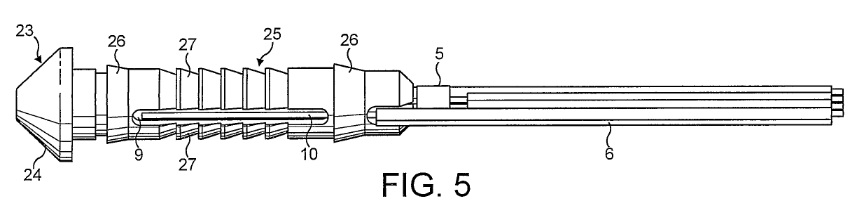

FIG. 5 is a view of the pCO₂ sensor of FIG. 3 with the membrane removed;

FIG. 6 illustrates a variant of the pCO₂ sensor of FIG. 3 wherein the attachment mechanism is visible;

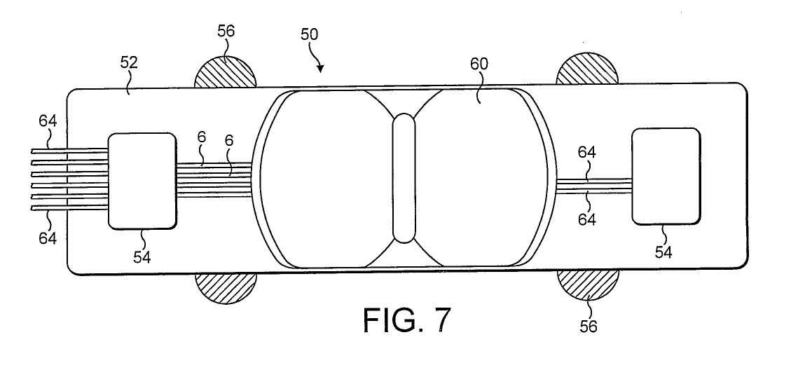

FIG. 7 is a plan view of a sensor device according to an embodiment of the invention;

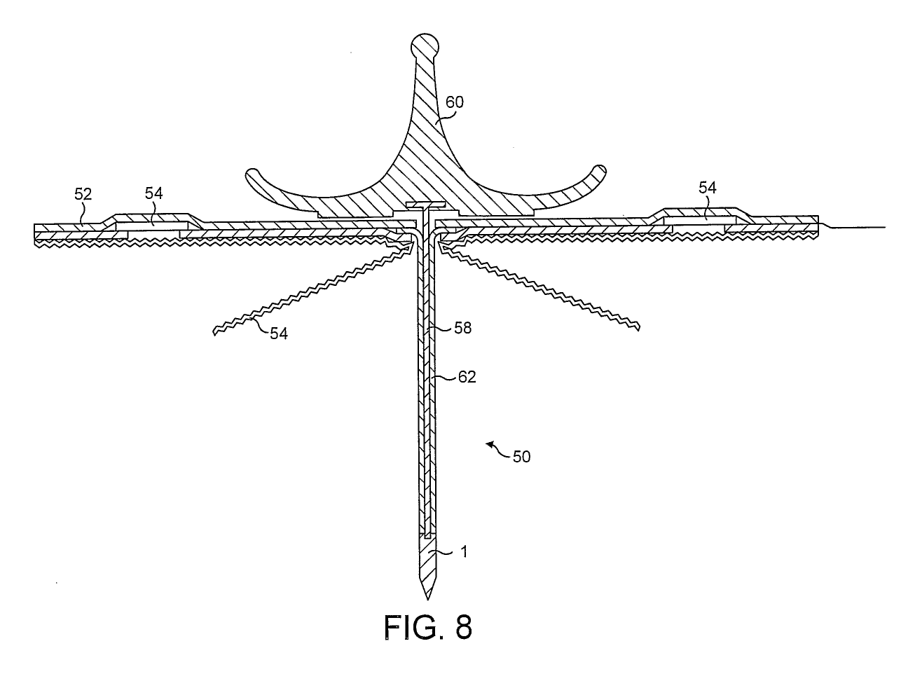

FIG. 8 is a side view, partially in section, of the sensor device of FIG. 7;

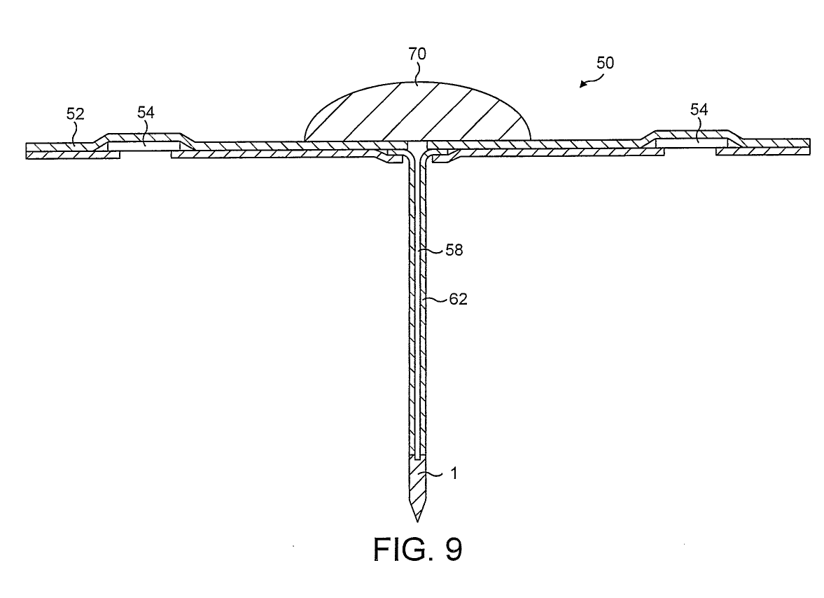

FIG. 9 is a side view of the sensor device of FIGS. 7 and 8 in the position of use;

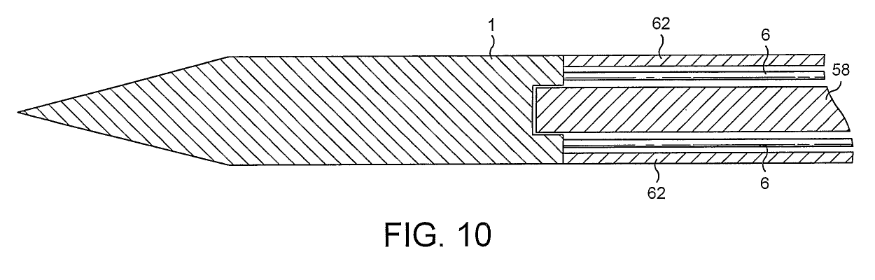

FIG. 10 is an enlarged view of the pCO₂ and temperature sensor of the sensor device of FIGS. 7 to 9;

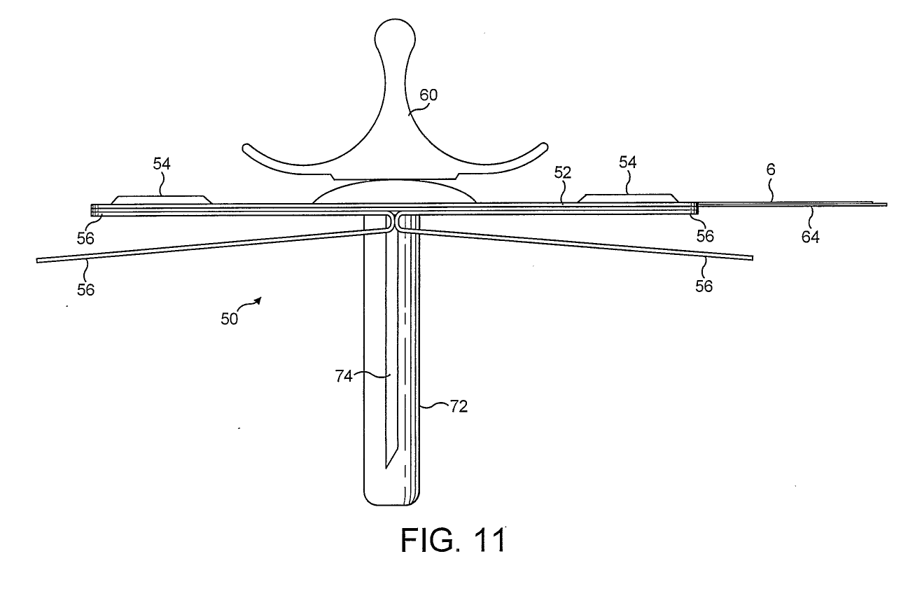

FIG. 11 shows a sensor device according to an alternative embodiment of the invention;

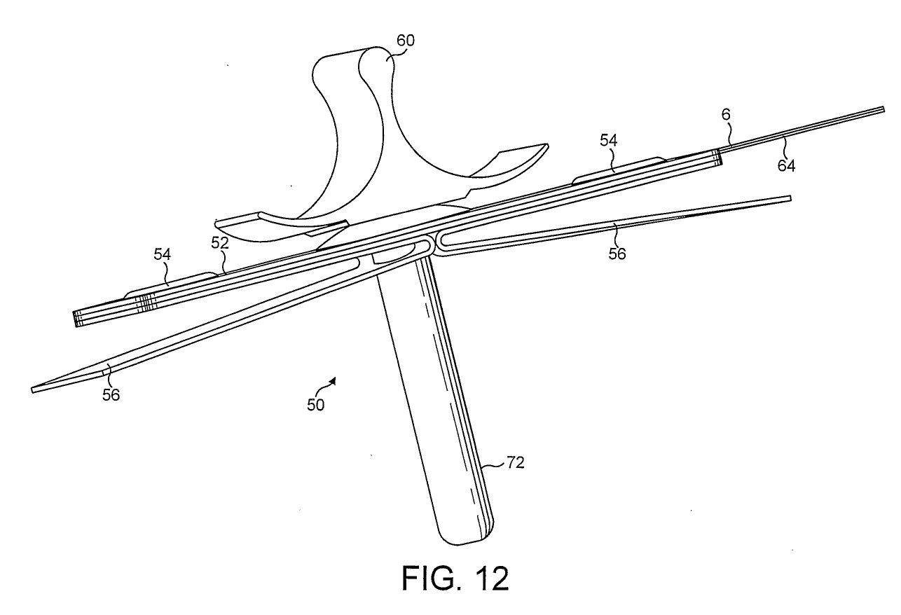

FIG. 12 is a perspective view, partially in section, of the sensor device of FIG. 11;

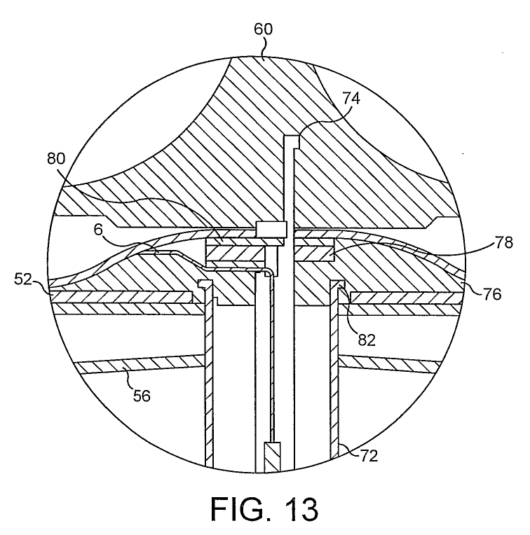

FIG. 13 is a sectional view of a details of the sensor device of FIGS. 11 and 12;

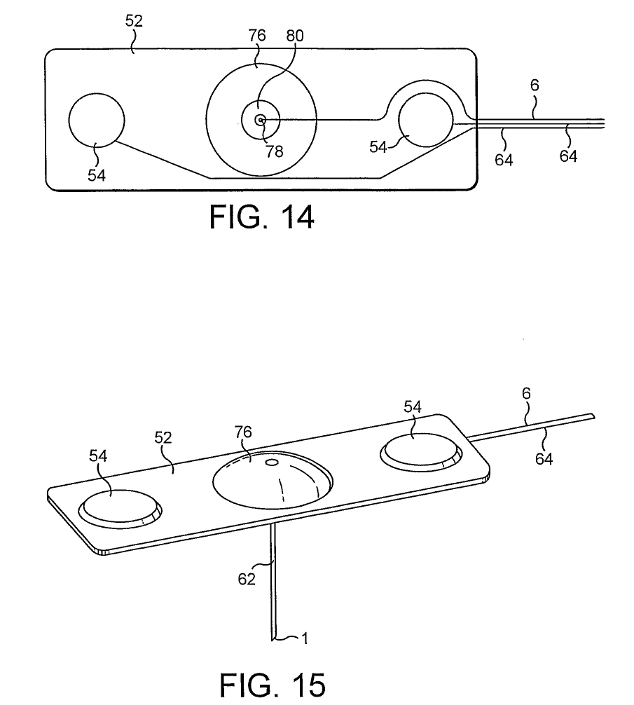

FIG. 14 is a plan view of the sensor device of FIGS. 11 to 13 without the insertion needle; and

FIG. 15 is a perspective view of the sensor device in the position of FIG. 14.

In accordance with the invention, a pCO₂ sensing system comprises a sensor device 50, an electronic surface unit 2, and a monitor unit 3, as shown in FIG. 1. The sensor device 50 comprises a combined pCO₂ and temperature sensor unit 1 and two pulse oxymetry sensors 54.

FIGS. 7 to 10 show the sensor device 50 according to an embodiment of the invention. The device 50 comprises a self-adhesive strip 52 onto which are mounted two reflection pulse oxymetry sensors 54 and a sensor unit 1 which will be described in detail below. The pulse oxymetry sensors may be of the type commercially available from Nellcor of Pleasanton, Calif. as MAX FAST adhesive forehead sensors. The self-adhesive strip 52 is provided with a release strip 56 which can be peeled from the adhesive strip 52 to reveal the adhesive surface of the adhesive strip 52 for application to a patient's skin. The sensor device 50 is provided packaged with the sensor unit 1 in a tube (not shown) filled with a sterile aqueous isotonic solution of propylene glycol to prevent any damage, contamination or evaporation.

The sensor device 50 includes a mandrel 58 provided with a finger grip 60. The mandrel 58 is received in a flexible sheath (or catheter) 62 which contains the cable connections 6 from the sensor unit 1. As shown in FIG. 10, at its distal end, the mandrel 58 engages the sensor unit 1 and allows the pointed sensor unit 1 to be driven through a patient's skin by the application of manual pressure to the finger grip 60 of the mandrel 58. In this way, the sensor unit 1 is located in the patient's muscle, for example in the patient's underarm.

When the pCO₂ sensor unit 1 has been located correctly in the patient's muscle, the mandrel 58 is withdrawn from the flexible sheath 62 leaving the sensor device 50 in the configuration shown in FIG. 9. The sheath 62 and cables 6 that are connected to the sensor unit 1 are sufficiently flexible that the patient feels little, if any, discomfort with the sensor unit 1 in position.

The sensor unit 1 is held in position in the muscle by the adhesive strip 52 adhering to the patient's skin. At the same time, the adhesion of the adhesive strip 52 to the skin brings the pulse oxymetry sensors 54 into their position of use against the patient's skin. The pulse oxymetry sensors 54 measure the reflectance of specified wavelengths of light from the patient's skin in order to determine the oxygen saturation level in the patient's blood.

As shown most clearly in FIG. 7, electrical connections 64 from the pulse oxymetry sensors 54 and from the sensor unit 1 run longitudinally along the adhesive strip 52 for connection to the electronic surface unit 2. Alternatively, as shown in FIG. 9, the sensor device 50 may be provided with a wireless device 70 for communication with the electronic surface unit 2 or the monitor unit 3.

The sensor device 50 is delivered packaged and sterilised. It includes a membrane-protected conductometric sensor 4 with a diameter of less than 1 millimetre, and a temperature probe 5 integrated in the sensor unit 1. Wires 6 connect the sensor 4 and probe 5 electrically by means of a connector to the electronic surface unit 2.

The electronic surface unit 2 sends and receives signals to and from the sensor device 50. It is placed on the patient's skin, performs signal processing on signals from the sensor unit 1 and transmits the conditioned signal to the monitor unit 5.

The monitor unit 3 is based on a portable personal computer 7 with PCMCIA input/output card 8 and Labview software (available from National Instruments Corporation of Austin, Tex.).

The pCO₂ sensor 4 is used for measurements of the level (partial pressure) of CO₂ (pCO₂) in tissue, according to the measurement principle illustrated in FIG. 2. The measurement chamber consists of two small cavities 9 with one electrode 10 positioned in each. The two cavities 9 are connected by one or more passageways 11 enclosed by a semi-permeable membrane 12, i.e. a membrane that only allows transport of CO₂ in and out of the volume of the sensor 4. The whole volume is filled with de-ionised water and 5% propylene glycol. The conductivity in the water depends upon the pCO₂, and by measuring the conductivity between the electrodes 10 in the volume, information about pCO₂ may be extracted.

As shown in FIGS. 3 to 5, the sensor unit 1 comprises an injection moulded plastics support 23, which is substantially cylindrical and surrounded by the semi-permeable membrane 12. The support 23 has a pointed tip 24 at its distal end and a body portion 25 which extends proximally from the tip 24. On the body portion 25 are mounted, by gluing, two gold electrodes 10. The electrodes 10 extend longitudinally along opposed sides of the body portion 25 and are received in respective recesses in the body portion 25.

Between the tip 24 and the body portion 25, a frustoconical projection 26 is provided for securing the membrane 12 by frictional fit. A corresponding projection 26 is provided at the proximal end of the body portion 25. The membrane 12 may be glued to the support 23, but it is important that the glue used to secure the membrane 12 and electrodes 10 is selected such that it does not bleed ions into the water-filled chamber formed between the body portion 25 of the support 23 and the membrane 12. Furthermore, the sealing faces of the support 23 may be made selectively hydrophobic in order to avoid the formation of a water film into which ions may bleed.

The membrane 12 may also be secured to the support 23 by means of crimp connection and a soft gasket, if necessary. The membrane 12 may act as the gasket, particularly where the membrane 12 is formed of silicone rubber. A heat shrink sleave may be used to form the crimp connection, as is the case in FIG. 6. Alternatively, metal crimp rings may be used in locations corresponding to those of the sealing projections 26.

The body portion 25 of the support 23 is provided with a plurality of ribs 27, which are formed with a saw tooth profile for easy moulding. The ribs 28 provide mechanical support to the membrane 12 and also define the fluid passageways 11 required for the sensor 4 to function effectively. Between each electrode 10 and the fluid passageways formed between the ribs 27 is provided a reservoir 9 formed by the recess in which the electrode 10 is located. The reservoir 9 provides a region of relatively low current density around the electrodes 10 in order to reduce electropolarisation effects.

During manufacture, the membrane 12 is fixed onto the support 23, while immersed in the de-ionised water and propylene glycol solution, so that the chamber bounded by the membrane 12, the electrodes 10, and the ribs 27 is completely filled with liquid. Thus, this chamber forms a pCO₂ sensor as shown schematically in FIG. 2.

It is possible for the sensor 1 to include more than one sensing chamber. For example, two parallel electrodes 10 separated by a wall member may be provided on each side of the support 23. A sensing chamber is thereby formed between one electrode 10 on one side of support 23 via the fluid passageways 11 between the ribs 27 on the top of the support 23 to one of the electrodes 10 on the other side of the support 23. A corresponding sensing chamber is provided between the remaining electrodes 10 and the fluid passageways 11 on the bottom of the support 11. An electrode 10 from each of these chambers may be electrically connected to the corresponding electrode from the other chamber, such that the electrical signal from the sensor reflects the conductivity of both chambers.

Embedded in the proximal end of the support 23 is a temperature sensor 5 in the form of a thermocouple. The temperature sensor 5 is used both for pCO₂ corrective calculations and for the measured tissue temperatures to be displayed on the monitor 3, which is informative for medical diagnosis. The temperature sensor 5 has a minimum measuring range of 33-42° C. and a minimum accuracy of +/−0.2° C.

A ribbon cable 6 is electrically and mechanically connected to the electrodes 10 and the temperature sensor 5. The electrodes 10 are formed as extensions of the conductors of the ribbon cable 6. Alternatively, the electrodes may be formed by plating onto the support 23. Where the cable 6 and the connection to the support 23 are sufficiently strong, the cable 6 can be used to pull the sensor unit 1 from its position of use. Alternatively, a Kevlar line may be provided, for example incorporated with the ribbon cable 6, to provide a strong external mechanical connection.

The membrane 12 may extend proximally from the support 23 with the cable 6 to form a catheter around the cable 6. Alternatively, a separate catheter 28 may be provided. In this case, the catheter 28 is bonded to the support 23 proximally of the electrodes 10 and the membrane 12.

As shown in FIG. 6, the catheter 28 may be provided with a plurality of slits 29 in order to fix the sensor unit 1 in position in tissue. The slits 29 are arranged such that when the catheter 28 is pushed distally (in the direction of the arrow B in FIG. 6), relative to the cable 6 (or Kevlar line) the portions 30 of the catheter 28 between the slits 29 are forced outwardly and assume the shape shown in phantom in FIG. 6. The radially projecting portions 30 of the catheter 28 retain the sensor unit 1 in the tissue in which it is embedded. The relative position of the catheter 28 and the cable 6 can be maintained with a locking mechanism (not shown) until it is time for the sensor unit 1 to be removed from the tissue. At this time, the locking mechanism can be released and the portions 30 of the catheter 28 will return to their relaxed position so that the sensor unit 1 can be removed from the tissue.

The catheter tip with the integrated sensor 4 is placed 0.5-4 cm into tissue to measure pCO₂ to detect and monitor the effect of treatment of the diseases and conditions mentioned above during a period of up to four weeks.

The sensor unit 1 has a maximum diameter of 1 mm and the maximum distance from the catheter tip to the sensor element is 2 mm. The sensor 4 has a minimum pCO₂ measuring range of 2-25 kPa, with a minimum detectable pCO₂ difference of 0.2 kPa. The maximum response of the sensor 4 is 20 seconds. The maximum allowable measurement current is in any area of the fluid chamber is such that j<1 mA/cm² while the measuring input voltage is not more than 50 mV RMS.

The electrodes 10 are gold plated and their total area is approximately 0.3 mm². The measurement frequency fₘₑₐₛ should be higher than 100 Hz. At lower frequencies, polarisation effects in the measurement chamber dominate the measurements. At frequencies above 10 kHz, the low impedance of the capacitances become a significant issue. The measurement resistance R_{—measure} is in the range of 500 kOhm to 7 MOhm.

The sensor 4 is electrically connected to an electronic surface unit 2 located on the patient skin by the ribbon cable 6, which has a length between 5 cm and 1 metre. The maximum diameter of the cable/catheter is 1 mm. The cable/catheter is soft and flexible so that it does not excessively disturb the neighbouring tissue. The cable/catheter and its connections are also sufficiently robust to withstand any pulling forces which may be caused by both normal and “abnormal” use.

During sterilisation, storage and transport the sensor unit 1 is covered by deionised, sterile and endotoxin-free water to make sure that there is substantially no net loss of water from the sensor reservoir.

FIGS. 11 to 15 show a sensor device 50 according to an alternative embodiment of the invention. Except where otherwise indicated, the configuration of this embodiment is the same as that of the sensor device described in relation to FIGS. 7 to 10. As in the previous embodiment, the device 50 comprises a self-adhesive strip 52 onto which are mounted two reflection pulse oxymetry sensors 54 and a sensor unit 1 as described above. The self-adhesive strip 52 is provided with a release strip 56 which can be peeled from the adhesive strip 52 to reveal the adhesive surface of the adhesive strip 52 for application to a patient's skin. The sensor device 50 is provided packaged with the sensor unit 1 in a sterile water-filled tube 72 filled with a sterile aqueous isotonic solution of propylene glycol to prevent any damage, contamination or evaporation.

The sensor device 50 includes a U-section insertion needle 74 provided with a finger grip 60. In the packaged sensor device 50, the sensor unit 1 and the associated cable connections are received in the U-shaped channel in the insertion needle 74. With the protective tube 72 removed, the insertion needle 74 can be driven through a patient's skin by the application of manual pressure to the finger grip 60. The insertion needle 74 can then be removed from the sensor device 50 leaving the sensor unit 1 located in the patient's muscle in the general configuration shown in FIG. 14. The U-shape of the insertion needle 74 allows the needle to be disengaged from the cable connections 6 to the sensor unit 1 as it is withdrawn.

FIG. 13 shows the detail of the connections between the insertion needle 74 and the sensor device 50. As shown in FIG. 13, the U-section insertion needle 74 is moulded into the finger grip 60. The sensor device 50 is provided with a plastic housing 76 which is located over and engages with an orifice defined in the self-adhesive strip 52. The plastic housing 76 is bonded to the self-adhesive strip 52. In the centre of the plastic housing 76 is defined a hole through which the insertion needle 74 passes. Above the hole in the plastic housing 76 a metal guide 78 in the form of a disc with a central hole for the insertion needle 74 is bonded to the plastic housing 76. The central hole in the metal guide 78 has a U-shape corresponding to the cross-section of the insertion needle 74 and acts to hold the needle 74 in position so that it cannot rotate and cause damage to the cable connections 6 to the sensor unit 1. The cable connections 6 from the sensor unit 1 pass from the insertion needle 74 between the metal guide 78 and the plastic housing 76 and are surrounded by a protective sheath 62 which is glued to the metal guide 78. The holes through the metal guide 78 and the plastic housing 76 are closed by a silicone membrane 80 provided over the metal guide and through which the insertion needle 74 passes. The silicone membrane 80 elastically deforms to seal the holes when the insertion needle 74 is removed.

As shown in FIG. 13, a beaded rim 82 of the cover tube 72 snap fits into a corresponding recess in the plastic housing 76 to seal the tube 72 to the sensor device 50. The tube 72 is removed from the sensor device 50 to expose the insertion needle 74 when the sensor unit 1 is to be inserted in the patient's muscle.

As shown in FIGS. 1 and 2, the electronic surface unit 2 comprises a sine generator 13 which provides a voltage of at least 5 Volts and a current supply of 50 mV, and is powered by batteries 14. A filter 15 is provided for filtering or averaging the input of the lock-in amplifier 16. A passive filter can be used which reduces the current consumption. A pre-amplifier 17 is combined with a servo mechanism to remove DC current from the signal to reduce electrolysis effects. According to the servo arrangement, the output of the pre-amplifier is fed back to its input via a low pass filter. Thus, only DC components of the output are fed back and cancel any DC current drawn through the pCO₂ sensor. In this way, it is ensured that there is no DC current through the pCO₂ sensor which would degrade the electrodes. The op-amp used in this stage consumes minimal current and has a large CMMR value. At the same time, the bias current is minimal. A lock-in amplifier 16 amplifies the AC signal from the sensor 4. This may be built with op-amps or using an IC package with at least 1% accuracy for the signal detection at frequencies lower than 1 kHz. A galvanic division 19 such as an optocoupler or a coil coupler is provided to prevent noise transfer from the monitor unit 3 and associated cabling 18. The optocoupler is normally favoured due to the noise signal ratio. A temperature signal amplification and conditioning unit 20 is provided to amplify the signal from the temperature sensor 5. The electronic unit 2 is powered by a rechargeable and changeable standard type battery 14. The battery capacity is sufficient for 14 days continuous monitoring. The surface unit 2 is also provided with an on/off indicator LED 21, and a battery status indicator (not shown). Communication between the surface unit 2 and the monitor 3 is analogue through a shielded cable 18. However, the surface unit 2 may include an analogue to digital converter such that communication between the surface unit 2 and the monitor 3 may be digital, for example by digital wire transmission or digital wireless transmission. The cable 18 is at least 4 m long and light and flexible.

As shown in FIGS. 1 and 2, an AC current is generated by sine generator 13 and fed to one of the pCO₂ sensor electrodes 10 and to a lock-in amplifier 16. The high-pass signal from the other pCO₂ electrode 10 is passed through a filter 15 to a low noise amplifier 17 and from there to the lock-in amplifier 16 where it is compared to the reference signal generated by the sine generator 13. Out of phase components, i.e. undesired components, of the signal are rejected and the remaining portion of the signal is amplified. The amplified signal is proportional to pCO₂ (or conductance) and is passed on for recordal or further manipulation to the monitor 3.

The surface unit 2 may also be electrically connected to a reference electrode (not shown) that is electrically connected to the patients skin. The signal from the reference electrode can be used to compensate the signals from the sensor unit 1 for the effect of electromagnetic noise generated by the patient.

A single surface unit 2 may receive signals from several sensor units 1 and provide a multiplexed output to the monitor unit 3.

The monitor unit 3 comprises a portable PC 7 including CD RW and IR port, and a PCMCIA I/O card 8 which can collect signals from at least 4 different surface units 2 simultaneously. The PCMCIA card 8 may have an integrated non-galvanic coupling. The power supply 22 for the monitor unit 3 is of a medically approved type operating on both 110V and 230V.

The software functions of the monitor unit 3 may be implemented in Labview, a software package available from National Instruments of Austin, Tex. and capable of handling up to 4 different surface units simultaneously. The software provides the facility for calibration of the sensor(s) with three calibration points and a second order calibration function. The software can be modified to support any other number of calibration points and type of calibration function. The software also has the facility to smooth the signal from the sensor device 50 over defined time intervals. It is possible to have at least two alarm levels for the measurement values and two alarm levels for their gradients. The measurement value gradients are calculated for individually defined time intervals. The alarm is both visible and audible. It is possible to stop an alarm indication while keeping the other alarms active. The monitor 3 can log all measured values, parameter settings and alarms throughout a session. With a 30 second logging interval there should be a storage capacity for at least 10 two week sessions on the hard disc. The session log can be saved to a writeable CD in a format readably by Microsoft Excel.

The sensor device 50 according to this embodiment of the invention is able to provide, in a single device, measurement of pCO₂, temperature and blood oxygenation of the patient's muscle. With this information, a physician can identify, amongst other conditions, the onset of sepsis in the patient quickly and accurately.

Although the sensor device has been described herein with particular reference to the measurement of pCO₂, the general configuration of the sensor device may be used for other physiological sensors, for example body temperature, partial pressure of oxygen, pH or glucose concentration.

1. A physiological sensing device comprising in combination:
a sensor for the measurement of the partial pressure of carbon dioxide (pCO2);
a body temperature sensor;
a heart rate sensor; and;
an oxygen saturation sensor.

2. A sensing device as claimed in claim 1, wherein the pCO2 sensor is configured for insertion through a patient's skin.

3. A sensing device as claimed in claim 1, wherein the temperature sensor is configured for insertion through a patient's skin.

4. A sensing device as claimed in claim 1, wherein the temperature sensor and the pCO2 sensor are provided by a sensor unit for insertion through a patient's skin.

5. A sensing device as claimed in claim 2 wherein the device comprises a sharp tip for puncturing a patient's skin on insertion of the pCO2 sensor.

6. A physiological sensing device comprising
a pCO2 sensor configured for insertion through a patient's skin and
a sharp tip for puncturing a patient's skin on insertion of the pCO2 sensor.

7. A sensing device as claimed in claim 6, wherein the sharp tip is provided by a removable hollow needle in which the pCO2 sensor is located for insertion through a patient's skin.

8. A sensing device as claimed in claim 1, wherein the oxygen saturation sensor is configured for application to the surface of a patient's skin.

9. A sensing device as claimed in claim 8, wherein the heart rate sensor and the oxygen saturation sensor are provided by a pulse oxymetry sensor.

10. A sensing device as claimed in claim 1 comprising an adhesive patch for adhering the device to a patient's skin.

11. A physiological sensing device comprising
a pCO2 sensor configured for insertion through a patient's skin and
an ablest patch for adhering the device to a patient's skin to retain the inserted pCO2 sensor in position.

12. A sensing device as claimed in claim 1, wherein the pCO2 sensor comprises a chamber bounded, at least in part, by a carbon dioxide permeable membrane and containing a substantially electrolyte-free liquid and at least two electrodes.

13. A sensing device as claimed in claim 6, wherein the pCO2 sensor comprises a chamber bounded, at least in part, by a carbon dioxide permeable membrane and containing a substantially electrolyte-free liquid and at least two electrodes.

14. A sensing device as claimed in claim 11, wherein the pCO2 sensor comprises a chamber bounded, at least in part, by a carbon dioxide permeable membrane and containing a substantially electrolyte-free liquid and at least two electrodes.

15. A sensing device as claimed in claim 5, wherein the sharp tip is provided by a removable hollow needle in which the pCO2 sensor is located for insertion through a patient's skin.

16. A sensing device as claimed in claim 6 comprising an adhesive patch for adhering the device to a patient's skin.
